(19) United States
(12) Patent Application Publication
(10) Pub. No.: US 2026/0201300 A1
(43) Pub. Date: Jul. 16, 2026

(54) MOBILE, MODULAR DOWNSTREAM PROCESSING SYSTEM FOR RECOVERY OF BIOMANUFACTURING PRODUCTS IN THE FIELD

(71) Applicant: Battelle Memorial Institute, Columbus, OH (US)

(72) Inventors: Jacob L. LILLY, Dublin, OH (US); Logan DANIELS, Columbus, OH (US); Julia WOOD, Westerville, OH (US); Caleb HILLRICH, Columbus, OH (US); Ryan DALY, Columbus, OH (US); Slawomir WINECKI, Dublin, OH (US)

(21) Appl. No.: 19/445,717

(22) Filed: Jan. 12, 2026

Related U.S. Application Data
(60) Provisional application No. 63/744,447, filed on Jan. 13, 2025.

(51) Int. Cl. C12M 3/00; C12M 1/00; C12M 1/02; C12M 1/34; C12M 1/36

(52) U.S. Cl. CPC C12M 23/44; C12M 23/52; C12M 29/06; C12M 29/14; C12M 41/18; C12M 41/40; C12M 41/48; C12M 45/04; C12M 45/06

(57) ABSTRACT

In an approach to mobile, modular downstream recovery of biomanufacturing products, a system includes a controller; and a filtration module; the system configured to: receive a feed stream; and filter the feed stream to produce a crude biomass product.

### CROSS-REFERENCE TO RELATED APPLICATIONS

The present application claims the benefit of the filing date of U.S. Provisional Application Serial No. 63/744,447, filed January 13, 2025, the entire teachings of which application is hereby incorporated herein by reference.

### STATEMENT REGARDING FEDERALLY SPONSORED RESEARCH OR DEVELOPMENT

This invention was made with government support under contract number HR001120C0118 awarded by the Defense Advanced Research Projects Agency. The government has certain rights in the invention.

### TECHNICAL FIELD

The present disclosure relates generally to product recovery and, more particularly, to a system and method for mobile, modular downstream recovery of biomanufacturing products in the field.

### BACKGROUND

Biomanufacturing of critical products in the field or in far remote locations continues to be a focal point for military, space, and polar missions. However, there are significant challenges to implementing these approaches. Biomanufacturing through cellular fermentation requires significant amounts of water, which needs to be separated from output products, but ideally also recovered to limit consumables and resupply requirements. Separation of water through heating or centrifugation requires significant energy input or large equipment that is difficult to transport or scale. There exists a need for a low size, weight, and power system that removes fermentation media from cellular products with drastically less energy than traditional equipment.

### BRIEF DESCRIPTION OF THE DRAWINGS

Reference should be made to the following detailed description which should be read in conjunction with the following figures, wherein like numerals represent like parts.

FIG. 1 is a functional block diagram illustrating an illustrative example system for mobile, modular downstream recovery of biomanufacturing products in the field consistent with the present disclosure.

FIG. 2 is a functional block diagram illustrating an illustrative example system of the filtration module from the example of FIG. 1, consistent with the present disclosure.

FIG. 3 is a graph of permeate flux versus filtration percentage for one example of the filtration module of FIG. 2, consistent with the present disclosure.

FIG. 4 is a perspective view of one illustrative example embodiment of the filtration module of FIG. 2, consistent with the present disclosure.

FIG. 5 is a functional block diagram illustrating an illustrative example system of the extraction module from the example of FIG. 1, consistent with the present disclosure.

FIG. 6 is a perspective view of one illustrative example embodiment of the extraction module of FIG. 2, consistent with the present disclosure.

FIG. 7 is a perspective view of one illustrative example embodiment of a scraper impeller for the extraction module of FIG. 5, consistent with the present disclosure.

FIG. 8 is a perspective view of one illustrative example embodiment of a formulation/transesterification column from the example of FIG. 1, consistent with the present disclosure.

FIG. 9 is a flowchart diagram depicting operations for the process for mobile, modular downstream recovery of biomanufacturing products in the field, on the example system of FIG. 1, consistent with the present disclosure.

FIG. 10 depicts a block diagram of components of the controller executing the process within the illustrative example system for mobile, modular downstream recovery of biomanufacturing products in the field of FIG. 1, consistent with the present disclosure.

### DETAILED DESCRIPTION

Disclosed herein is a low size, weight, and power system that removes fermentation media from cellular products with drastically less energy than traditional equipment. The system is also versatile to allow downstream refining, such as solvent extraction or chemical modification. Each unit operation is modular and can be scaled independently or removed if not needed for a particular use case. The disclosed system may process biomanufacturing outputs, such as biomass from fermenters, primarily in austere environments. Characteristics of the system may include, but are not limited to, modularity, low size, weight, power, and cost (SWAP-C), automatable controls, minimum consumables, reconfigurability, transportability, and low maintenance.

Disclosed herein is a series of integrated subcomponents that work in concert to form a system for biomanufactured product recovery. The disclosed system is particularly well suited for recovering intracellular or whole cell biomass products after fermentation but can be reconfigured to also recover extracellular products. The system can be applied for recovery of a wide range of final products including lipid products, food or nutraceutical products, and/or chemical precursors. The system can also be applied to food biomanufacturing wherein the organism itself is edible but requires dehydration.

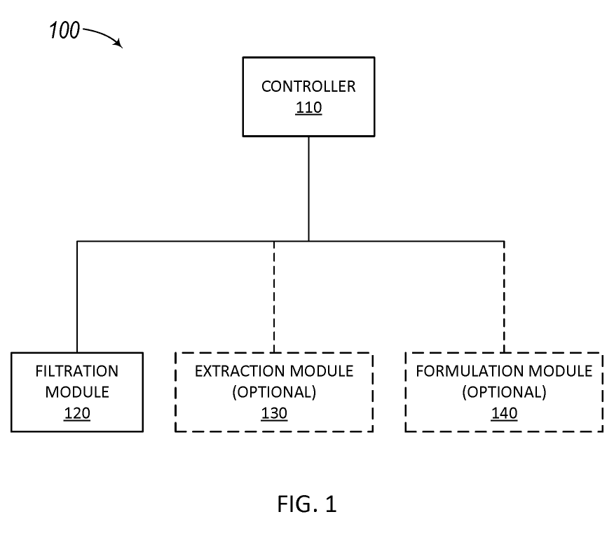

FIG. 1 is a functional block diagram illustrating an illustrative example system 100 for mobile, modular downstream recovery of biomanufacturing products in the field consistent with the present disclosure. The example system 100 includes a controller 110, a filtration module 120, an optional extraction module 130, and an optional formulation module 140. These modules are described in more detail in the following paragraphs.

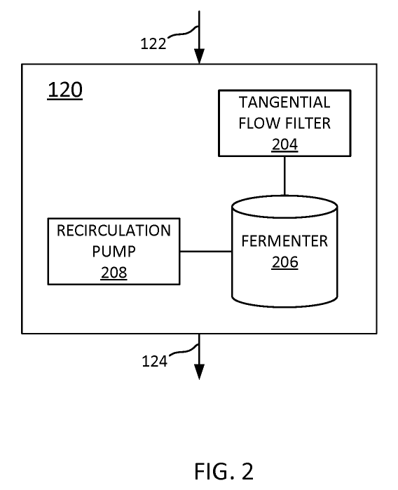

FIG. 2 is a functional block diagram illustrating an illustrative example of the filtration module 120 from FIG. 1, consistent with the present disclosure. The filtration module is based on a reusable size exclusion filter (e.g., a Tangential Flow Filter (TFF) 204) in-line with a recirculation loop and connected to a one or more fermenters 206 that serves as the vessel for biomanufacturing, where a recirculation pump 208 moves the fermentation slurry across the TFF 204. Excess water content and water soluble compounds are removed from the dilute feed stream 122 using the TFF 204. The TFF 204 utilizes a porous membrane and flow generated by the recirculation pump 208 to retain large particles (e.g., microbial cells) while allowing smaller molecules to pass through the membrane. This method of filtration creates very low shear forces compared to normal flow filtration (e.g., pressurized filtration), allowing cell walls to remain intact. This process utilizes only pump energy to drive dehydration, and is a more power efficient method of bulk water removal than thermal evaporative methods. In this process, a biological culture is pumped through a cartridge in the TFF 204. The pressure and flow created by the pump and cartridge drives small particles and molecules like water, salts, and cell culture media to permeate through the membrane pores. Cell culture media separated from cells can be recovered and reused in subsequent fermentation runs. The desired biological products are retained and recirculated through the system to remove as much water as possible. The membrane chemistry (poly ether sulfone or modified polyether sulfone in this example process) is chosen to ensure chemical compatibility with the feed stream. Filter area is chosen based on batch size, desired processing time, and pump requirements. For example, a larger membrane area is suited for larger processes which have a high flow rate pump. An industrial process size would also be less affected by the large hold up volume of a larger membrane.

A smaller process size may sacrifice processing time in order to yield more product (as less product is retained in the filter hold up volume) and to use less energy, since lower surface area cartridges require lower pump flow rates. The filter pore size is selected to ensure retention of target molecules. For example, the pore size for a retentate product should be 2-5 times smaller than the target molecule. A 500 to 750 kilodalton (kDa) pore size is ideal for bacterial cell concentration. In an embodiment, additional process operating parameters, which may include, but are not limited to, transmembrane pressure (pressure difference between the feed/retentate side vs the permeate side of the cartridge) and pump flow rate, are controlled to achieve the desired shear stress and bulk water removal rate. The set points for these parameters may be determined through optimization experiments, as feed stream contents can have varying effects. For bacterial culture concentration, a high pump flow rate may increase the shear rate, reducing the gel layer build up on the filter membrane.

In one illustrative example operation, 25 liters of biological culture may be reduced to 10% or less of the original volume, or to a concentration of 5-10% cell dry weight. A TFF filter, such as those manufactured by Repligen Corporation of Waltham, Massachusetts, with modified polyether sulfone membrane (mPES) is preferred. In this example, an mPES membrane was selected as the membrane is more hydrophilic than a polyether sulfone (PES) membrane, which exhibits improved filter performance with a high cell density feedstock. In this instance, improved performance of mPES refers to less gel layer formation, leading to less backpressure for the same or greater permeate flux compared to PES. A preliminary design utilized three PES cartridges in parallel (each with 0.042 square meters of filter area). A more matured design featured a single mPES cartridge (0.16 square meters of filtration area). Using one longer cartridge instead of three in parallel creates fewer connections and fewer potential leak points. Finally, the single mPES cartridge allowed for more secure tubing connections using tri-clamp fittings on all sides, while the three cartridges used tubing stubs on the permeate side. A shear rate of 12000 reciprocal seconds (s⁻¹), a transmembrane pressure of 50% of the filter membrane rating (15 psi), and a permeate flowrate to maintain approximately 30 flux LMH was determined to be optimal to balance gel layer formation, power efficiency, and processing time.

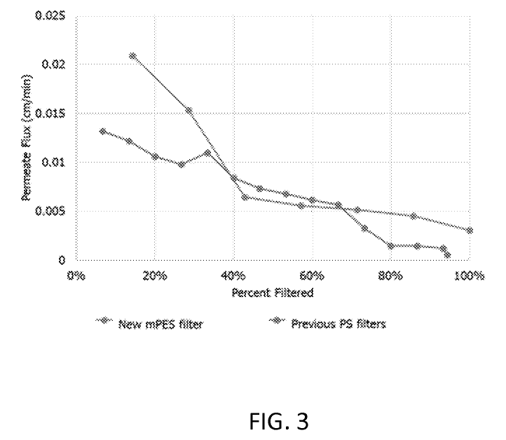

In one example, the TFF 204 performed dewatering over the course of approximately six hours, nominally to greater than 95% water removal with an mPES cartridge. A plot of permeate flux versus filtration percentage is shown in FIG. 3 comparing performance for three PES cartridges versus one mPES cartridge. This plot shows that the mPES design maintains higher flux near the end of the filtration run, which ultimately allows for more overall water to be removed.

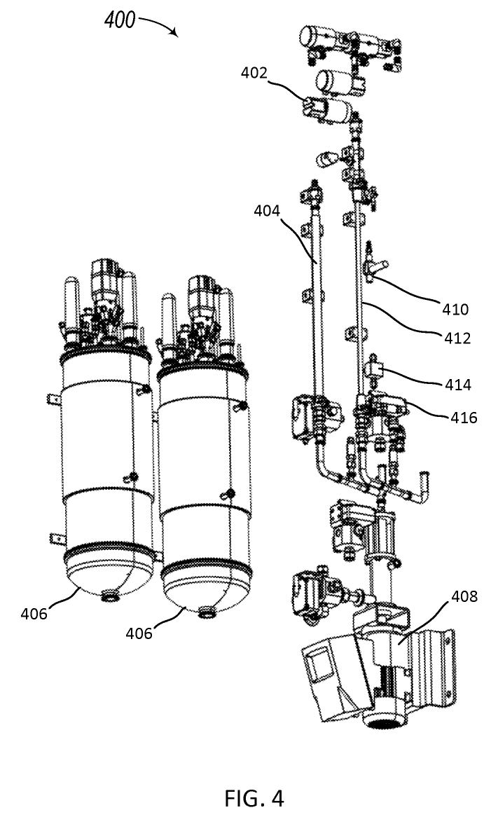

FIG. 4 is a perspective view of one illustrative example embodiment of the filtration module of FIG. 2, consistent with the present disclosure. The example of FIG. 4 includes recirculation valves 402 to control the recirculation of the permeate through the TFF 412, a bypass tube 404, one or more fermenters 406, a recirculation metering pump 408, a permeate control valve 410, a permeate flowmeter 414, and one or more pressure sensors 416.

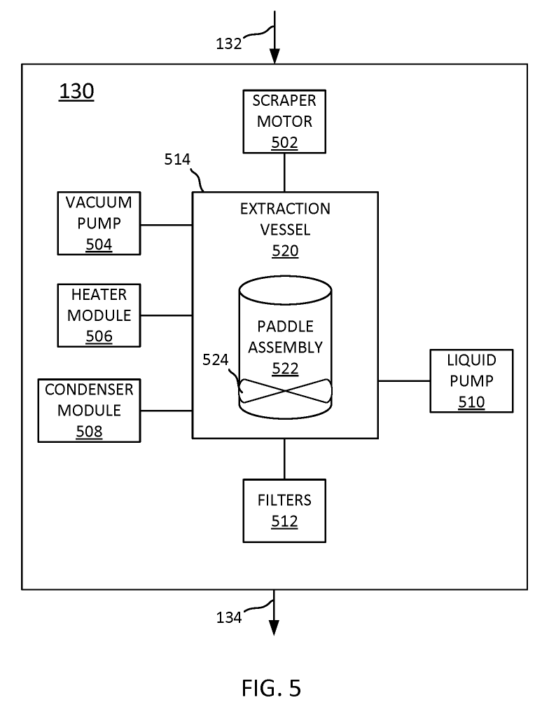

FIG. 5 is a functional block diagram illustrating an illustrative example of the extraction module 130 from the example of FIG. 1, consistent with the present disclosure. In the extraction module 130, a crude biomass product 132 is further concentrated through paddle scraping and vacuum-assisted dehydration to result in extraction output 134. In numerous processes, it is beneficial to remove as much water from the biomass as possible. This dehydration step is particularly critical in systems where a highly concentrated product is required, the formulation module is water sensitive, and/or when product stability is important. The disclosed extraction vessel 520 is specifically designed to address these requirements, facilitating efficient and effective dehydration of the biomass under controlled conditions.

The extraction module 130 contains the jacketed extraction vessel 520 with an inner paddle assembly 522. A heat transfer fluid fills the jacket 514 to ensure uniform heating. The heat transfer fluid may be heated by heater module 506. The paddle assembly 522 consists of a main shaft which is connected to the scraper motor 502 to turn the one or more paddles 524, and two symmetric spring-loaded shafts lined with scraper blades that contact the inner vessel wall. The main shaft is rotated to scrape the inner vessel wall to prevent buildup and char, while also facilitating heat and mass transfer. In an embodiment, spring-loaded shafts are used to apply adequate contact force for the scraping blades, but also for their ability to handle slight variations in the vessel wall and to keep the motor torque manageable. This inner paddle assembly is designed to mimic the scraping action found in many large, industrial sludge dryers, while maintaining the small scale necessary for the application. Scraper blades may be constructed of polyether ether ketone (PEEK) for abrasion and chemical resistance and is preferred over metal blades to reduce risk of sparking and explosion when operating in the presence of flammable solvents.

Also attached to the extraction vessel is a vacuum pump 504 which lowers the system pressure to reduce the boiling point of the water within the biomass slurry. By lowering the boiling point, the temperature required to evaporate water from the biomass is also reduced. This feature prevents the biomass from undergoing thermal degradation or burning. As water is evaporated from the biomass, it is recovered in a condenser module 508 and held for purification and recycling.

For whole cell products, such as in food biomanufacturing, the dried biomass can be emptied and collected for use. For recovery of intracellular products, the disclosed system can also act as an extraction vessel, wherein a solvent of choice, such as methanol, can be added to perform extraction of intracellular products. Optionally, a solvent such as ethanol may be added to form an azeotrope, which aids in removing additional water. During or after extraction, reactions can also be performed, such as transesterification of lipid products to alkyl esters (methyl esters, ethyl esters, etc.). Mixing during extraction can be achieved by activation of the paddles, or through pump-around mixing with an accompanying recirculation loop. To remove any residual cell solids or other debris, the extraction output 134 can also be routed in the recirculation loop to a filter 512, which may be an inline filter and/or a micron filter. If necessary, additional reactions to extract the desired product can be performed at this stage. Following these reactions, a process-compatible solvent is introduced into the extraction vessel to resuspend the biomass for transport to the formulation module by liquid pump 510.

In one illustrative example operation for the conversion and extraction of fatty acid methyl esters from biomass, one liter (L) of a 5-10 weight percent cell solids slurry is added to the extraction vessel 520. The jacket 514 is then heated to 65°C, the vacuum pump 504 applies 27 inches of Mercury (inHg) of vacuum, and the scraper motor 502 is activated at 5 revolutions per minute (RPM). Once the condenser module 508 contains enough water mass to suggest sufficient biomass drying (approximately 70% cell dry weight), a base-catalyzed extraction and esterification of triglycerides is performed inside the vessel. This reaction uses a methanol solvent and is catalyzed by sodium methoxide. Upon reaction completion, the product is pumped through a one micron filter to remove cell debris and returned to the extraction vessel for methanol removal. The methanol is removed in the same manner as the previous dehydration step. Lastly, the extraction product is resuspended in hexane to remove salts and filtered with an in-line 0.2 micron filter en route to the formulation module.

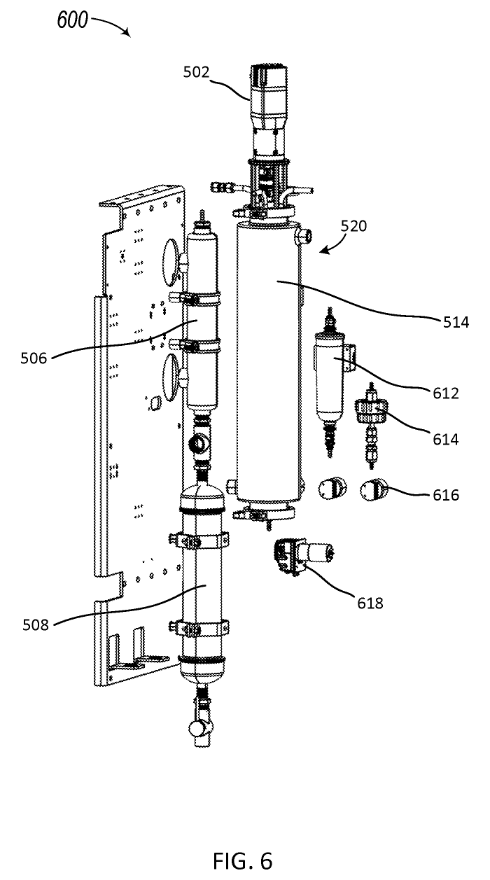

FIG. 6 is a perspective view of one illustrative example embodiment of the extraction module 130 of FIG. 5, consistent with the present disclosure. The example of FIG. 6 includes the scraper motor 502, a vacuum pump 504 (not shown), the heater module 506, the condenser module 508, the jacketed extraction vessel 520 including the jacket 514, the filters which may include a coarse filter 612 and/or a fine filter 614, one or more valves 616, and the extraction liquid pump 510.

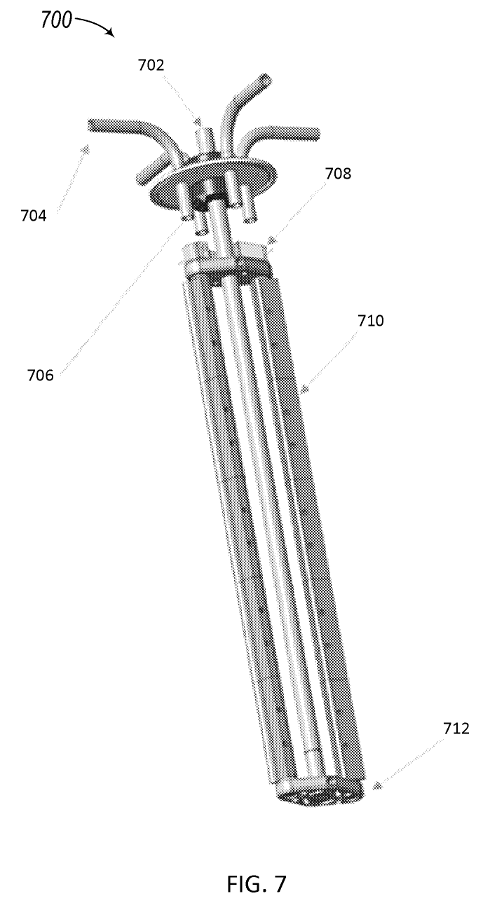

FIG. 7 is a perspective view of one illustrative example embodiment of a scraper impeller for the extraction module of FIG. 5, consistent with the present disclosure. The example of FIG. 7 includes motor shaft 702, which connects, for example, to scraper motor 502, one or more inlet/outlet tubes 704, one or more bearings and seals 706, one or more scraper springs 708 as discussed above in FIG. 5, one or more scraper blades 710 (the example of FIG. 7 includes ten scraper blades 710), and a bottom scraper 712.

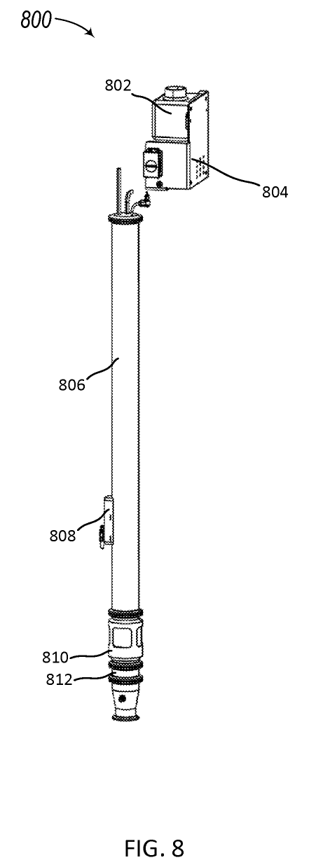

FIG. 8 is a perspective view of one embodiment of an optional formulation/transesterification column from the example of FIG. 1, consistent with the present disclosure. Depending on the use and product to be extracted, additional chemical modifications or blending may be desired. In the illustrative example embodiment of FIG. 8, the formulation module consists of a sparging column 806 that can optionally be heated via a jacket or by an electric heater (not shown). The column contains a fritted disc 812 and an air sparging pump 808 to enable air sparging through the fritted disc 812, but the fritted disc 812 also allows liquid product to be drained while keeping solid particulate within the column, such as catalyst resin, for reuse. Sparging can promote evaporation of residual solvent or mix the contents without the need for an impeller and motor. A chemical supply tank 802 and a chemical supply liquid pump 804 may be connected to the sparging column 806 to enable addition of reagents or additives to a product for formulation.

In one illustrative example operation for the production of a bio-based lubricant, the fatty acid methyl ester product recovered from a biomass is transesterified in the formulation module to create a higher viscosity product for use as a lubricant. The resuspended filtered lipids are pumped into the transesterification vessel. Air is bubbled through the mixture using the connected air sparging pump to remove the solvent. Once sufficient solvent is removed, 2-ethyl hexanol and Novozyme 435 lipase enzyme beads 810 are added to the column, which continues to be bubbled with air. The Novozyme 435 lipase enzyme beads 810 allow for efficient transesterification of lipids without added heat, at just 2% weight per volume loading to the estimated mass of fatty acid methyl esters (FAMEs) from extraction. The bubbling promotes mixing and the evaporation of solvents until the product reaches sufficient concentration.

In another illustrative example, Rhodococcus opacus is fermented in the one or more fermenters 206, wherein the bacteria accumulate triglyceride oils intracellularly. The fermenter contents are then pumped through the TFF 204 and recirculated though the one or more fermenters 206 until the product is dehydrated to 5-10% cell solids. This concentrated cell slurry exits filtration module 120 as extraction output 134. The material stream 124 enters extraction module 130 as stream 122 and is added the extraction vessel 520. The heater module 506 heats the heat transfer fluid to 65°C to circulate through the jacketed extraction vessel 520, and the vacuum pump 504 applies 27 inHg of vacuum. The scraper motor 502 is activated at five RPM to dry the concentrated slurry. Once dried, a base-catalyzed extraction and esterification of triglycerides is performed inside the extraction vessel 520 to form solubilized fatty acid methyl esters. This reaction uses a methanol solvent and is catalyzed by sodium methoxide. Upon reaction completion, the product is pumped through a one micron filter to remove cell debris and returned to the extraction vessel for methanol removal. The methanol is removed in the same manner as the previous dehydration step. Lastly, the extraction product is resuspended in hexane to remove salts and filtered with an in-line 0.2 micron filter 512 en route to the formulation module.

In the formulation module 140, methyl esters can be further dried and used as biofuel, or further transesterified with a branched alcohol, like 2-ethyl hexanol, with the sparging column 806 and a resin-bound lipase enzyme catalyst. Other additives can also be added into the sparging column 806, such as antioxidants, viscosity modifiers, stabilizers, etc. After formulation, the liquid product can be drained out of the column for collection and use.

In another illustrative example, a biomanufacturing strain is grown in the one or more fermenters 206 that produces precursors or products for use as fuels, solvents, cleaner, adhesives, or polymer resin, which are dehydrated in the filtration module 120 and extracted in the extraction module 130 in a similar fashion as described above.

In another illustrative example, mixtures of dicarboxylic acids derived from the oxidation of polyolefin waste are dissolved in acetone and added directly to formulation module 140. A molar excess of 2-3-fold 2-ethyl hexanol is added to the sparging column 806. Dicarboxylic acids are directly esterified with 2-ethyl hexanol and a resin-bound lipase catalyst. Air sparging in the formulation module 140 drives evaporation of acetone solvent while also mixing the reaction. Once complete, liquid aliphatic 2-ethyl-hexyl bis-esters that have desired viscosity and lubricant properties are drained from the column through the fritted disc 812 for use.

In another illustrative example, multiple fermenters 206 are controlled by controller 110 with staggered operation to achieve a semi-continuous fermentation crude product output that feeds a common extraction module and/or formulation module to increase overall production rate.

In another illustrative example, desired extracellular products that are produced within the one or more fermenters 206 can be recovered through the cartridges in the TFF 204, wherein the soluble products permeate the TFF membrane, while cells are returned to the one or more fermenters 206 for continued fermentation or to serve as a seed for future fermentation runs. The permeate containing soluble products are transferred to extraction module 130 and extraction vessel 520 wherein the product can be dehydrated for recovery. Examples of these products are proteins and chemical precursors.

In another illustrative example, edible microbial strains can be fermented in the one or more fermenters 206 for biomanufacturing food. After fermentation, microbes are concentrated within the TFF 204 and collected as a paste, or transferred to the extraction vessel 520 within extraction module 130 wherein the whole cell product is dehydrated for collection as consumption as a dried clay-like product.

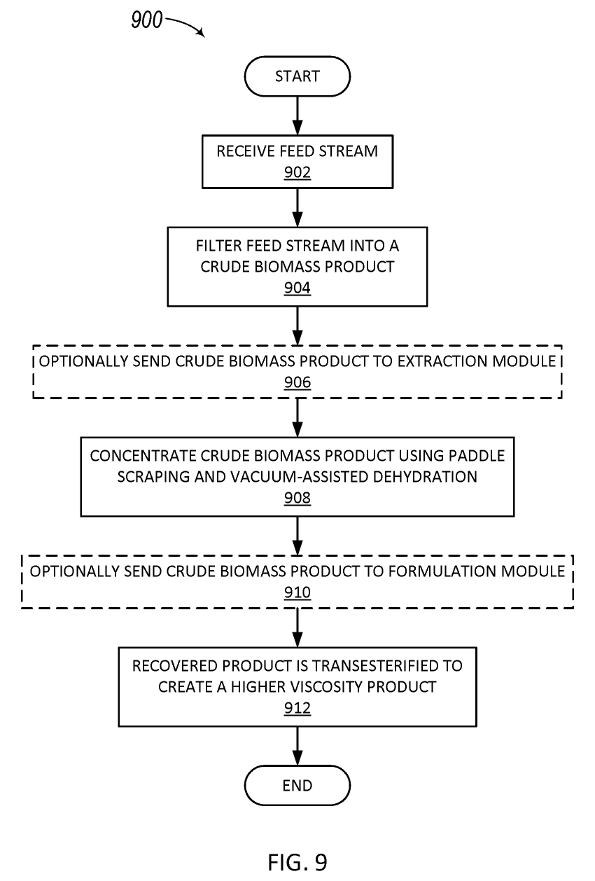

FIG. 9 is a flowchart diagram depicting operations for the process for mobile, modular downstream recovery of biomanufacturing products in the field, on the example system of FIG. 1, consistent with the present disclosure. It should be appreciated that embodiments of the present disclosure provide at least for mobile, modular downstream recovery of biomanufacturing products in the field. However, FIG. 9 provides only an illustration of one implementation and does not imply any limitations with regard to the environments in which different embodiments may be implemented. Many modifications to the depicted environment may be made by those skilled in the art without departing from the scope of the disclosure as recited by the claims.

Receive a feed stream (operation 902). In the illustrated example embodiment, a feed stream for recovery of biomanufacturing products is input to the filtration module.

Filter the feed stream into a crude biomass product (operation 904). The feed stream is filtered, e.g., dewatered, using one or more filters, such as TFF 204 from FIG. 2. Excess water content and water soluble compounds are removed from the dilute feed stream and the result is a crude biomass product.

Send the crude biomass product to an extraction module (optional operation 906). If further concentration of the biomass product is desired, then in optional operation 906 the crude biomass product is sent to an extraction module, such as extraction module 130 from FIG. 1.

Concentrate the crude biomass product using paddle scraping and vacuum‐assisted dehydration (operation 908). The extraction module further concentrates the crude biomass received in operation 906 through paddle scraping and vacuum-assisted dehydration to generate an extraction output. A jacket covering the extraction vessel is filled with a heat transfer fluid to maintain a constant temperature to ensure uniform heating. A paddle assembly consisting of a main shaft connected to a scraper motor to turn the paddles and two symmetric spring-loaded shafts lined with scraper blades is rotated to scrape the inner vessel wall to prevent buildup and char, while also facilitating heat and mass transfer.

Send the crude biomass product to a formulation module (optional operation 910). Depending on the intended use of the extraction product, additional chemical modifications or blending may be desired. If additional chemical modifications or blending is desired, then in optional operation 910 the biomass product resulting from operation 904 or the extraction output from operation 908 is sent to a formulation/transesterification module, such as formulation module 140 from FIG. 1.

Recovered product is transesterified to create a higher viscosity product (operation 912). In operation 912, the product recovered from the biomass in operation 904 or operation 908 is further processed, e.g., transesterified, in the formulation module to create a higher viscosity product for use as, for example, a lubricant.

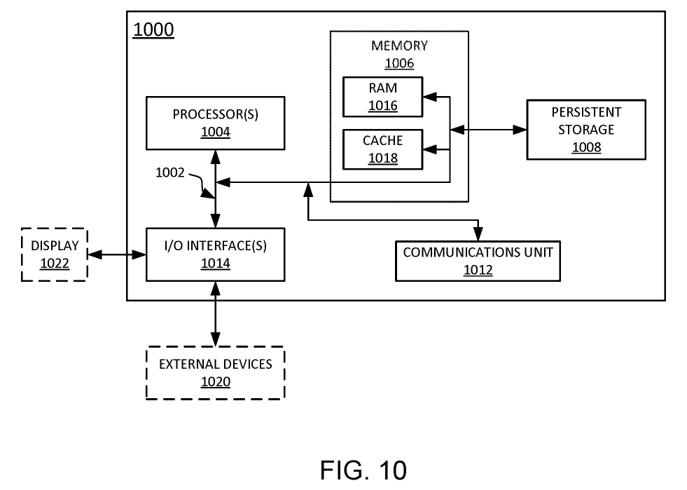

FIG. 10 is a block diagram depicting components of one example of the controller 110 from FIG. 1 suitable for mobile, modular downstream recovery of biomanufacturing products in the field, within the illustrative example system of FIG. 1. FIG. 10 displays the controller or computer 1000, one or more processor(s) 1004 (including one or more controllers or computer processors), a communications fabric 1002, a memory 1006 including, a random-access memory (RAM) 1016 and a cache 1018, a persistent storage 1008, a communications unit 1012, I/O interfaces 1014, a display 1022, and external devices 1020. It should be appreciated that FIG. 10 provides only an illustration of one embodiment and does not imply any limitations with regard to the environments in which different embodiments may be implemented. Many modifications to the depicted environment may be made.

As depicted, the computer 1000 operates over the communications fabric 1002, which provides communications between the computer processor(s) 1004, memory 1006, persistent storage 1008, communications unit 1012, and input/output (I/O) interface(s) 1014. The communications fabric 1002 may be implemented with an architecture suitable for passing data or control information between the processors 1004 (e.g., microprocessors, communications processors, and network processors), the memory 1006, the external devices 1020, and any other hardware components within a system. For example, the communications fabric 1002 may be implemented with one or more buses.

The memory 1006 and persistent storage 1008 are computer readable storage media. In the depicted embodiment, the memory 1006 comprises a RAM 1016 and a cache 1018. In general, the memory 1006 can include any suitable volatile or non-volatile computer readable storage media. Cache 1018 is a fast memory that enhances the performance of processor(s) 1004 by holding recently accessed data, and near recently accessed data, from RAM 1016.

Program instructions for mobile, modular downstream recovery of biomanufacturing products in the field may be stored in the persistent storage 1008, or more generally, any non-transitory computer readable storage media, for execution by one or more of the respective computer processors 1004 via one or more memories of the memory 1006. The persistent storage 1008 may be a magnetic hard disk drive, a solid-state disk drive, a semiconductor storage device, flash memory, read only memory (ROM), electronically erasable programmable read-only memory (EEPROM), or any other computer readable storage media that is capable of storing program instruction or digital information.

The media used by persistent storage 1008 may also be removable. For example, a removable hard drive may be used for persistent storage 1008. Other examples include optical and magnetic disks, thumb drives, and smart cards that are inserted into a drive for transfer onto another computer readable storage medium that is also part of persistent storage 1008.

The communications unit 1012, in these examples, provides for communications with other data processing systems or devices. In these examples, the communications unit 1012 includes one or more network interface cards. The communications unit 1012 may provide communications through the use of either or both physical and wireless communications links. In the context of some embodiments of the present disclosure, the source of the various input data may be physically remote to the computer 1000 such that the input data may be received, and the output similarly transmitted via the communications unit 1012.

The I/O interface(s) 1014 allows for input and output of data with other devices that may be connected to computer 1000. For example, the I/O interface(s) 1014 may provide a connection to external device(s) 1020 such as a keyboard, a keypad, a touch screen, a microphone, a digital camera, and/or some other suitable input device. External device(s) 1020 can also include portable computer readable storage media such as, for example, thumb drives, portable optical or magnetic disks, and memory cards. Software and data used to practice embodiments of the present disclosure can be stored on such portable computer readable storage media and can be loaded onto persistent storage 1008 via the I/O interface(s) 1014.

I/O interface(s) 1014 may also connect to a display 1022. Display 1022 provides a mechanism to display data to a user and may be, for example, a computer monitor. Display 1022 can also function as a touchscreen, such as a display of a tablet computer.

According to one aspect of the disclosure there is thus provided a system for mobile, modular downstream recovery of biomanufacturing products, the system including: a controller; and a filtration module; the system configured to: receive a feed stream; and filter the feed stream to produce a crude biomass product.

According to yet another aspect of the disclosure, there is provided a method of mobile, modular downstream recovery of biomanufacturing products, the method including: receiving a feed stream; and filtering the feed stream to produce a crude biomass product.

Although the methods and systems have been described relative to a specific embodiment thereof, they are not so limited. Obviously, many modifications and variations may become apparent in light of the above teachings. Many additional changes in the details, materials, and arrangement of parts, herein described and illustrated, may be made by those skilled in the art. Also, it may be appreciated that the phraseology and terminology used herein is for the purpose of description and should not be regarded as limiting as such may be understood by one of skill in the art. Throughout the present disclosure, like reference characters may indicate like structure throughout the several views, and such structure need not be separately discussed. Furthermore, any particular feature(s) of a particular exemplary embodiment may be equally applied to any other exemplary embodiment(s) of this disclosure as suitable. In other words, features between the various exemplary embodiments described herein are interchangeable, and not exclusive.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

“Circuitry,” as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry and/or future computing circuitry including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DSP), field programmable gate array (FPGA), logic gates, registers, semiconductor device, chips, microchips, chip sets, etc.

The term “coupled" as used herein refers to any connection, coupling, link, or the like by which signals carried by one system element are imparted to the "coupled" element. Such “coupled" devices, or signals and devices, are not necessarily directly connected to one another and may be separated by intermediate components or devices that may manipulate or modify such signals.

Unless otherwise stated, use of the word "substantially" may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles "a" and/or "an" and/or "the" to modify a noun may be understood to be used for convenience and to include one, or more than one, of the modified noun, unless otherwise specifically stated. The terms "comprising", "including" and "having" are intended to be inclusive and mean that there may be additional elements other than the listed elements.

What is claimed is:

1. A system for mobile, modular downstream recovery of biomanufacturing products, the system comprising:
a controller; and
a filtration module;
the system configured to:
receive a feed stream; and
filter the feed stream to produce a crude biomass product.

2. The system of claim 1, wherein the filtration module further comprises:
one or more tangential flow filters;
one or more fermenters; and
a recirculation pump, the filtration module further configured to:
recirculate a fermentation slurry across the one or more tangential flow filters to remove excess water content and water soluble compounds from the feed stream.

3. The system of claim 2, wherein a filter pore size of the one or more tangential flow filters is 2-5 times smaller than a target molecule for a retentate product.

4. The system of claim 2, wherein a filter pore size of the one or more tangential flow filters is 500 to 750 kilodaltons for bacterial cell concentration.

5. The system of claim 1, further comprising:
an extraction module, the extraction module configured to:
receive the crude biomass product from the filtration module; and
concentrate the crude biomass product into an extraction output.

6. The system of claim 5, wherein the extraction module further comprises:
a scraper motor;
a vacuum pump configured to lower a system pressure to reduce a boiling point of water within the crude biomass product;
a heater module;
a liquid pump; and
an extraction vessel, the extraction vessel further comprising:
a jacket filled with heat transfer fluid to ensure uniform heating;
a paddle assembly; and
one or more paddles, the extraction vessel further configured to:
concentrate the crude biomass product into the extraction output through paddle scraping and vacuum-assisted dehydration.

7. The system of claim 5, wherein:
the crude biomass product is concentrated using paddle scraping and vacuum-assisted dehydration.

8. The system of claim 5, wherein a solvent is added to form an azeotrope to aid in removing additional water.

9. The system of claim 8, wherein the solvent is ethanol.

10. The system of claim 5, wherein the extraction output is routed to an inline filter to remove residual cell solids or other debris.

11. The system of claim 6, further comprising:
a formulation module, the formulation module configured to:
receive the extraction output from the extraction module; and
apply additional chemical modifications to the extraction output.

12. The system of claim 11, wherein the formulation module further comprises:
a chemical supply tank;
a chemical supply liquid pump;
a sparging column;
an air sparging pump; and
one or more fritted discs, the formulation module further configured to:
create a higher viscosity product by transesterification of an extraction output.

13. A method for mobile, modular downstream recovery of biomanufacturing products, the method comprising:
receiving a feed stream; and
filtering the feed stream to produce a crude biomass product.

14. The method of claim 13, further comprising:
sending the crude biomass product to an extraction module; and
concentrating the crude biomass product.

15. The method of claim 14, wherein:
the crude biomass product is concentrated using paddle scraping and vacuum-assisted dehydration.

16. The method of claim 13, further comprising:
sending the crude biomass product to a formulation module; and
creating a higher viscosity product using transesterification.
